SEQUENCE LISTING (27 sequences)

SEQ ID NO: 1 - protein, 8 aa, Artificial Sequence
  Synthetic peptide
QHTQITKV

SEQ ID NO: 2 - protein, 8 aa, Artificial Sequence
  Synthetic peptide
QHSQITKV

SEQ ID NO: 3 - protein, 8 aa, Artificial Sequence
  Synthetic peptide
  MISC_FEATURE (3)..(3): X denotes Ser or Thr
  MISC_FEATURE (6)..(6): X denotes Ser or Thr
  MISC_FEATURE (8)..(8): X denotes Val, Leu or Ile
QHXQIXKX

SEQ ID NO: 4 - protein, 13 aa, Artificial Sequence
  Synthetic peptide
PFDEDQHTQITKV

SEQ ID NO: 5 - protein, 12 aa, Artificial Sequence
  Synthetic peptide
FDEDQHTQITKV

SEQ ID NO: 6 - protein, 11 aa, Artificial Sequence
  Synthetic peptide
DEDQHTQITKV

SEQ ID NO: 7 - protein, 10 aa, Artificial Sequence
  Synthetic peptide
EDQHTQITKV

SEQ ID NO: 8 - protein, 9 aa, Artificial Sequence
  Synthetic peptide
DQHTQITKV

SEQ ID NO: 9 - protein, 8 aa, Artificial Sequence
  Synthetic peptide
QHTQITKL

SEQ ID NO: 10 - protein, 8 aa, Artificial Sequence
  Synthetic peptide
QHTQITKI

SEQ ID NO: 11 - protein, 8 aa, Artificial Sequence
  Synthetic peptide
QHTQISKV

SEQ ID NO: 12 - protein, 8 aa, Artificial Sequence
  Synthetic peptide
QHTQISKL

SEQ ID NO: 13 - protein, 8 aa, Artificial Sequence
  Synthetic peptide
QHTQISKI

SEQ ID NO: 14 - protein, 13 aa, Artificial Sequence
  Synthetic peptide
PFDEDQHSQITKV

SEQ ID NO: 15 - protein, 12 aa, Artificial Sequence
  Synthetic peptide
FDEDQHSQITKV

SEQ ID NO: 16 - protein, 11 aa, Artificial Sequence
  Synthetic peptide
DEDQHSQITKV

SEQ ID NO: 17 - protein, 10 aa, Artificial Sequence
  Synthetic peptide
EDQHSQITKV

SEQ ID NO: 18 - protein, 9 aa, Artificial Sequence
  Synthetic peptide
DQHSQITKV

SEQ ID NO: 19 - protein, 8 aa, Artificial Sequence
  Synthetic peptide
QHSQITKL

SEQ ID NO: 20 - protein, 8 aa, Artificial Sequence
  Synthetic peptide
QHSQITKI

SEQ ID NO: 21 - protein, 8 aa, Artificial Sequence
  Synthetic peptide
QHSQISKV

SEQ ID NO: 22 - protein, 8 aa, Artificial Sequence
  Synthetic peptide
QHSQISKL

SEQ ID NO: 23 - protein, 8 aa, Artificial Sequence
  Synthetic peptide
QHSQISKI

SEQ ID NO: 24 - DNA, 19 bp, Artificial Sequence
  Synthetic oligonucleotide
caggtggtggagcaagatg

SEQ ID NO: 25 - DNA, 27 bp, Artificial Sequence
  Synthetic oligonucleotide
ccgagatctctgaagtgaagatggatg

SEQ ID NO: 26 - DNA, 30 bp, Artificial Sequence
  Synthetic oligonucleotide
cctctttgtgactatgtggactgatgtcgg

SEQ ID NO: 27 - DNA, 28 bp, Artificial Sequence
  Synthetic oligonucleotide
gtggataccccctcccccagcctagacc

(19) United States
(12) Patent Application Publication
(10) Pub. No.: US 2022/0153782 A1
(43) Pub. Date: May 19, 2022

(54) COMPOUNDS, COMPOSITIONS AND METHODS FOR TREATING A NEUROLOGICAL CONDITION

(71) Applicant: MEMORYPLUS LTD., Tel Aviv (IL)

(72) Inventor: Shira KNAFO, Beer Sheva (IL)

(21) Appl. No.: 17/437,628

(22) PCT Filed: Mar. 24, 2020
(86) PCT No.: PCT/IL2020/050345

§ 371 Date: Sep. 9, 2021

Related U.S. Application Data
(60) Provisional application No. 62/824,334, filed on Mar. 27, 2019.

(51) Int. Cl. C07K 7/06; A61P 25/28

(52) U.S. Cl. CPC C07K 7/06; A61P 25/28

(57) ABSTRACT

This invention relates to novel peptide conjugates, composition comprising the same, and uses thereof in the treatment of neurological conditions.

### SEQUENCE LISTING

The instant application contains a Sequence Listing which has been submitted electronically in ASCII format and is hereby incorporated by reference in its entirety. Said ASCII copy, created on Mar. 19, 2020, is named P-586107-PC-SQL-MAR20 ST25.txt and is 5,765 bytes in size.

### FIELD OF THE INVENTION

This invention relates to novel peptide-conjugates, composition comprising the same, and uses thereof in the treatment of neurological conditions.

### BACKGROUND OF THE INVENTION

Gradual accumulation of amyloid-beta (Aβ) peptide induces a series of synaptic and neuronal dysfunctions, which are considered to be responsible for cognitive deficits ranging in severity from mild-cognitive impairment (MCI) to Alzheimer's dementia. Accumulating evidence indicates that soluble Aβ assemblies directly alter synaptic plasticity mechanisms by inhibiting long-term potentiation (LTP) and facilitating long-term depression (LTD) in hippocampal neurons. Therefore, it is believed that Aβ shifts the synaptic plasticity balance toward a pathologically enhanced form of depression.

Lipid phosphatase PTEN mediates depression. It is postulated that it binds to the PDZ domains of PDZ-proteins in neurons and such binding affects accumulation of Aβ and induces long-term depression.

Binding of a short peptide or a derivative thereof, e.g., a peptide conjugate, to the PDZ-binding site may prevent PTEN from binding to the same site (FIG. 1). As a result, PTEN cannot accumulate at the PDZ-binding site, and which, in turn, lowers the accumulation of amyloid-beta (Aβ) peptide, and prevents or treats conditions or diseases characterized by presence of accumulation or deposits of Aβ peptide aggregated to an insoluble mass in the brain of a patient, including Alzheimer's disease and long-term depression.

### SUMMARY OF THE INVENTION

In one embodiment, this invention provides novel synthetic peptide conjugates. The peptide conjugates are used for the treatment of neurological conditions. In one embodiment, this invention provides a medication comprising novel peptide conjugates. In one embodiment, this invention provides a process of treating a subject, the process comprising administering the novel peptide conjugates to the subject. In one embodiment, administering the peptide conjugates of this invention to a subject, improves, restores and/or preserve cognitive function.

In one aspect, the present invention provides a peptide conjugate N-Dodecanoyl-QHTQITKV (conjugate of peptide SEQ ID No: 1).

In one aspect, the present invention provides a peptide conjugate N-Dodecanoyl-QHSQITKV (conjugate of peptide SEQ ID No: 2).

In another aspect, the present invention further provides a method of preventing or treating a β-amyloidogenic disease comprising administering a pharmaceutically effective amount of a peptide conjugate of the invention, or a derivative or peptidomimetic thereof, to a subject in need thereof. In one embodiment, the β-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM).

In one aspect, the present invention further provides a method of preventing, mitigating, or alleviating synaptic or cognitive deficits associated with a β-amyloidogenic disease. In one embodiment, the β-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM).

In another aspect, the present invention provides a method of preventing or treating Alzheimer's disease comprising administering a pharmaceutically effective amount of a peptide conjugate of this invention, or a derivative or peptidomimetic thereof, to a subject in need thereof.

In another aspect, the present invention provides a method of treating symptoms of Alzheimer's disease comprising administering a pharmaceutically effective amount of a peptide conjugate of this invention, or a derivative or peptidomimetic thereof, to a subject in need thereof.

In one embodiment, the symptoms treated by the method of the invention are mild cognitive impairment or age-associated memory loss.

In one embodiment, this invention provides a method of enhancing cognitive function in healthy individuals.

In one embodiment, this invention provides memory enhancement/improvement for healthy individuals. In one embodiment, this invention provides a method for the enhancement of cognitive function for subjects with age-related cognitive impairment. In one embodiment, this invention provides a method for memory improvement.

In one embodiment, composition of this invention is administered by injection.

The present invention further provides a composition comprising a peptide conjugate and a pharmaceutically acceptable carrier.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically shows proposed mechanism for synapse recovery induced by administration of the PTEN-PDZ peptide derivative.

FIGS. 2A and 2B show analysis results for Dodecanoyl-QHSQITKV (conjugate of peptide SEQ ID 2); FIG. 2A HPLC analysis; FIG. 2B Mass spectrometry analysis.

FIGS. 3A and 3B show analysis results for Dodecanoyl-QHTQITKV (conjugate of peptide SEQ ID 1); FIG. 3A HPLC analysis; FIG. 3B Mass spectrometry analysis.

FIGS. 4A and 4B show the effect of the synthesized peptide conjugates on cognitive function; FIG. 4A shows performance in the Barnes maze; FIG. 4B shows results of a contextual fear conditioning test.

FIG. 5 shows the results of a contextual fear conditioning test comparing myristoyl derivative with dodecanoyl derivative.

FIG. 6 shows percentage of peptide remaining after incubation in plasma (mouse) FIG. 6A; plasma (human) FIG. 6B; brain/liver homogenates FIG. 6C and FIG. 6D; or in simulated intestinal fluid FIG. 6E. N, 3 independent experiments. Data are mean±SEM; Labels are presented in FIG. 6F.

FIG. 7A and FIG. 7B show cell metabolic activity (%) versus log concentration (μM) of peptides after 4 hours exposure (FIG. 7A) and after 24 hours exposure (FIG. 7B) (n=3). Data are Mean±SEM.

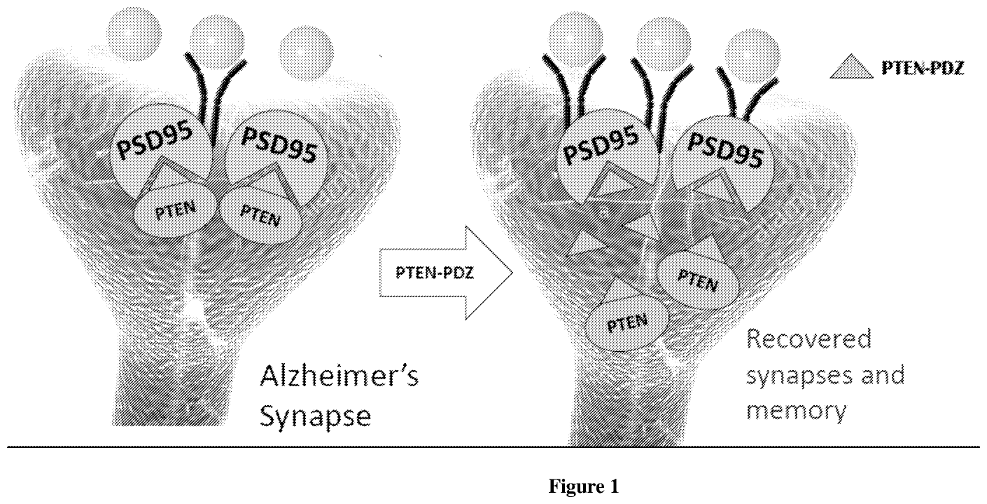

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one aspect, the present invention provides a peptide conjugate represented by Formula (I):

### Fatty Acid-Peptide (I)

In one embodiment, the fatty acid moiety is attached to the peptide moiety by forming an amide linkage. For example, the carboxylic group of the fatty acid reacts with an amino group of the peptide to form an amide linkage. In one embodiment, the fatty acid reacts with the amino group at the N-terminus of the peptide to form an amide linkage.

The fatty acid component of the peptide conjugate of the invention is typically a saturated or unsaturated C₈-C₂₄ hydrocarbon carboxylic acid, or mixtures thereof. Examples of suitable saturated fatty acids include, but are not limited to, caprylic acid (C₈H₁₆O₂), capric acid (C₁₀H₂₀O₂), undecylenic acid (C₁₁H₂₂O₂), lauric acid (C₁₂H₂₄O₂) (dodecanoic acid), myristic acid (C₁₄H₂₈O₂), palmitic acid (C₁₆H₄₄O₂), stearic acid (C₁₈H₃₆O₂), arachidic acid (C₂₀H₄₀O₂), behenic acid (C₂₂H₄₄O₂), and lignoceric acid (C₂₄H₄₈O₂), and mixtures thereof. In one embodiment, the fatty acid component of the peptide conjugate of the invention is not myristic acid (C₁₄H₂₈O₂). In one embodiment, peptide conjugates of this invention do not comprise the myristoyl peptide conjugate (myristoyl-QHSQITKV). In one embodiment, peptide conjugates of this invention do not comprise the myristoyl peptide conjugate (myristoyl-QHTQITKV).

Examples of suitable mono-unsaturated fatty acids include, but are not limited to, palmitoleic acid (C₁₆H₃₀O₂), oleic acid (C₁₈H₃₄O₂), ebidic acid (C₁₈H₃₄O₂), erucic acid (C₂₂H₄₂O₂), and brassidic acid (C₂₂H₄₂O₂), and mixtures thereof. Examples of suitable di- or tri-unsaturated fatty acids are linoleic acid (C₁₈H₃₂O₂) and linolenic acid (C₁₈H₃₀O₂), and mixtures thereof.

In one embodiment, the fatty acid is capric acid (C₁₀H₂₀O₂), undecylenic acid (C₁₁H₂₂O₂), or lauric acid (C₁₂H₂₄O₂) (dodecanoic acid). In one embodiment, the fatty acid is palmitic acid (C₁₆H₄₄O₂), stearic acid (C₁₈H₃₆O₂), arachidic acid (C₂₀H₄₀O₂), behenic acid (C₂₂H₄₄O₂), or lignoceric acid (C₂₄H₄₈O₂). In another embodiment, the fatty acid is lauric acid (C₁₂H₂₄O₂) (dodecanoic acid).

In one embodiment, the fatty acid component of the peptide conjugate of the invention is a saturated or unsaturated C₈-C₁₃ hydrocarbon carboxylic acid, or mixtures thereof.

In one embodiment, the term “peptide” denotes an amino acid polymer that is composed of at least two amino acids covalently linked by an amide bond. In one embodiment, the peptide contains 8 to 20 residues in length. In other embodiment, the peptide contains 8 to 16 residues in length. In certain embodiment, the peptide contains 8 to 10 residues in length. In one embodiment, the peptide contains 6 to 8 residues in length. In one embodiment, the peptide contains 6 to 20 residues in length. In one embodiment, the peptide contains 7 to 9 residues in length. In one embodiment, the peptide contains 8 to 13 residues in length. In one embodiment, the peptide contains 6 to 13 residues in length. In one embodiment, the peptide contains 8 residues in length.

In some embodiments, an inhibitor, e.g., the peptide-conjugate of the invention, that selectively blocks PDZ-dependent recruitment of PTEN comprises an 8 to 20 amino acid residue peptide, comprising or consisting of the amino acid sequence Gln-His-Xaa₁-Gln-Ile-Xaa₂-Lys-Xaa₃ (SEQ ID NO:3),

wherein

Xaa₁ is Ser or Thr,

Xaa₂ is Ser or Thr, or any residue in which there is a hydroxy group at the beta position, and

Xaa₃ is Val, Leu, or Ile, or any residue having an aliphatic side chain.

In one embodiment, Xaa₁ and Xaa₂ are independently Ser or Thr, and Xaa₃ is Val, Leu or Ile.

In one embodiment, Xaa₃ is Val, Leu or Ile.

In certain embodiments of the present invention, a selective inhibitor of the invention has an amino acid sequence as listed in Table 1.

TABLE 1

| Inhibitor Peptide Sequence | SEQ ID NO: |
|---|---|
| PFDEDQHTQITKV | 4 |
| FDEDQHTQITKV | 5 |
| DEDQHTQITKV | 6 |
| EDQHTQITKV | 7 |
| DQHTQITKV | 8 |
| QHTQITKV | 1 |
| QHTQITKL | 9 |
| QHTQITKI | 10 |
| QHTQISKV | 11 |
| QHTQISKL | 12 |
| QHTQISKI | 13 |
| PFDEDQHSQITKV | 14 |
| FDEDQHSQITKV | 15 |
| DEDQHSQITKV | 16 |
| EDQHSQITKV | 17 |
| DQHSQITKV | 18 |
| QHSQITKV | 2 |
| QHSQITKL | 19 |
| QHSQITKI | 20 |
| QHSQISKV | 21 |
| QHSQISKL | 22 |
| QHSQISKI | 23 |

Sequences 1 and 4-13, are equivalent or are based on or are similar to sequences found naturally as part of the C-terminus of human PTEN's. Sequences 2 and 14-23, are equivalent, or are based on or are similar to sequences found naturally as part of the C-terminus of mice PTEN's.

In one embodiment, the peptide moiety of the peptide conjugate of the invention is QHTQITKV (SEQ ID NO:1). In one embodiment, the peptide moiety is QHSQITKV (SEQ ID NO:2).

In one embodiment, when the fatty acid is dodecanoic acid, the fatty acid moiety of Formula (I) is Dodecanoyl. In one embodiment, the fatty acid is a derivative of Dodecanoyl.

In one embodiment, the peptide conjugate of the invention is N-Dodecanoyl-QHTQITKV (conjugate of peptide SEQ ID NO:1). In another embodiment, the peptide conjugate of the invention is N-Dodecanoyl-QHSQITKV (conjugate of peptide SEQ ID NO:2).

In one embodiment, the peptide conjugate of the invention is N-Dodecanoyl-QHTQITKV (conjugate of peptide SEQ ID NO:1). In another embodiment, the peptide conjugate of the invention is N-Dodecanoyl-QHSQITKV (conjugate of peptide SEQ ID NO:2). It is well known in the art, the “N” refers to Dodecanoyl being linked to the amino group of the peptide moiety.

In one embodiment, the peptide conjugate of the invention is represented by Formula (II)

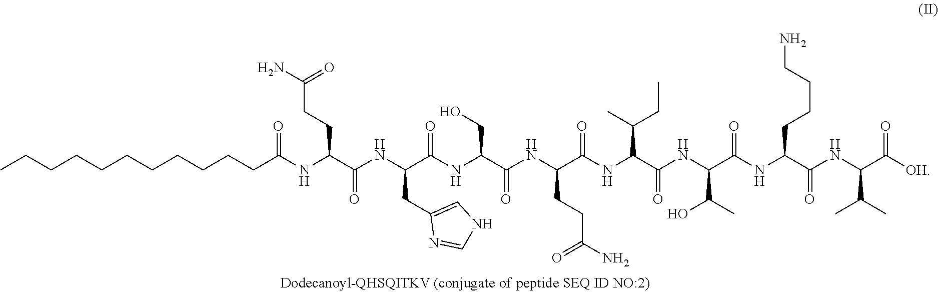 [SMILES: CCCCCCCCCCCC(=O)N[C@@H](CCC(N)=O)C(=O)N[C@H](Cc1c[nH]cn1)C(=O)N[C@@H](CO)C(=O)N[C@H](CCC(N)=O)C(=O)NC(C(=O)N[C@@H](C(=O)N[C@@H](CCCCN)C(=O)N[C@@H](C(=O)O)C(C)C)C(C)O)[C@@H](C)CC]

In one embodiment, the peptide conjugate of the invention is represented by Formula (III)

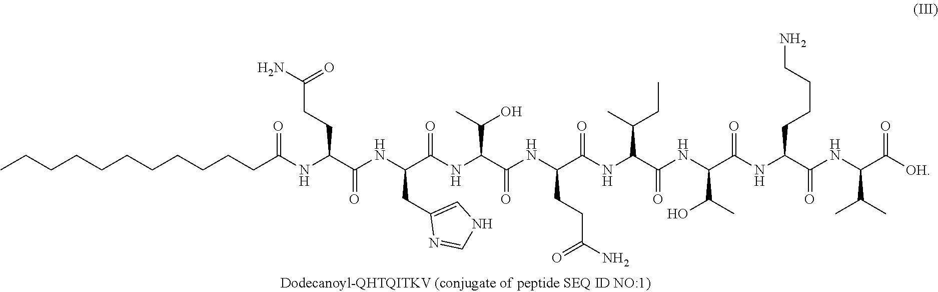 [SMILES: CCCCCCCCCCCC(=O)N[C@@H](CCC(N)=O)C(=O)N[C@H](Cc1c[nH]cn1)C(=O)N[C@H](C(=O)N[C@H](CCC(N)=O)C(=O)NC(C(=O)N[C@@H](C(=O)N[C@@H](CCCCN)C(=O)N[C@@H](C(=O)O)C(C)C)C(C)O)[C@@H](C)CC)C(C)O]

In some aspects, the present invention provides a peptide conjugate represented by Formula (IV)

### Cholesterol-Peptide

### IV

In one embodiment, the cholesterol moiety is linked to the peptide moiety via a carbamate bond between the N-terminal of the peptide and the OH group of the cholesterol. In another embodiment, the cholesterol is linked to any functional group of the side chains of the amino acids. All peptide sequences described herein as conjugated with fatty acids, can also be conjugated to cholesterol instead of a fatty acid in one embodiment, and all such cholesterol-peptide conjugates, compositions thereof and methods of use thereof as described herein with reference to peptide-fatty acid conjugates are included in this invention.

In one embodiment, the peptide moiety is a peptide derivative that is a molecule which retains the primary amino acids of the peptide, however, the N-terminus, C-terminus, and/or one or more of the side chains of the amino acids therein have been chemically altered or derivatized. Such derivatized peptides include, for example, naturally occurring amino acid derivatives, for example, allo-threonine, 4-hydroxyproline for proline, 5-hydroxylysine for lysine, homoserine for serine, ornithine for lysine, and the like. Other derivatives or modifications include, e.g., a label, such as fluorescein or tetramethylrhodamine; or one or more post-translational modifications such as acetylation, amidation, formylation, hydroxylation, methylation, myristoylation, palmitoylation, stearoylation, phosphorylation, sulfatation, glycosylation, or lipidation.

In addition, a peptide in the peptide conjugate of the invention can include a cell-penetrating sequence which facilitates, enhances, or increases the transmembrane transport or intracellular delivery of the peptide into a cell. For example, a variety of proteins, including the HIV-1 Tat transcription factor, Drosophila Antennapedia transcription factor, as well as the herpes simplex virus VP22 protein have been shown to facilitate transport of proteins into the cell (Wadia and Dowdy (2002) Curr. Opin. Biotechnol. 13:52-56). Further, an arginine-rich peptide (Futaki (2002) Int. J. Pharm. 245:1-7), a polylysine peptide containing Tat PTD (Hashida, et al. (2004) Br. J. Cancer 90(6):1252-8), Pep-1 (Deshayes, et al. (2004) Biochemistry 43(6):1449-57) or an HSP70 protein or fragment thereof (WO 00/31113) is suitable for enhancing intracellular delivery of a peptide or peptidomimetic of the invention into the cell.

In one embodiment, the peptide of the peptide-conjugate of the present invention also encompasses peptidomimetics of the peptides disclosed herein. Peptidomimetics refer to a synthetic chemical compound which has substantially the same structural and/or functional characteristics of the peptides of the invention. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy the activity of the mimetic. The phrase “substantially the same,” when used in reference to a mimetic or peptidomimetic, means that the mimetic or peptidomimetic has one or more activities or functions of the referenced molecule.

There are advantages for using a mimetic of a given peptide. For example, there are considerable cost savings and improved patient compliance associated with peptidomimetics, since they can be administered orally compared with parenteral administration for peptides. Furthermore, peptidomimetics can be cheaper to produce than peptides.

The techniques of developing peptidomimetics are conventional. For example, peptide bonds can be replaced by non-peptide bonds or non-natural amino acids that allow the peptidomimetic to adopt a similar structure, and therefore biological activity, to the original peptide. Further modifications can also be made by replacing chemical groups of the amino acids with other chemical groups of similar structure. The development of peptidomimetics can be aided by determining the tertiary structure of the original peptide by NMR spectroscopy, crystallography and/or computer-aided molecular modeling. These techniques aid in the development of novel compositions of higher potency and/or greater bioavailability and/or greater stability than the original peptide (Dean (1994) BioEssays 16:683-687; Cohen & Shatzmiller (1993) J. Mol. Graph. 11:166-173; Wiley & Rich (1993) Med. Res. Rev. 13:327-384; Moore (1994) Trends Pharmacol. Sci. 15:124-129; Hruby (1993) Biopolymers 33:1073-1082; Bugg, et al. (1993) Sci. Am. 269:92-98).

It will be readily apparent to one skilled in the art that a peptidomimetic can be generated from any of the peptides described herein. It will furthermore be apparent that the peptidomimetics can be further used for the development of even more potent non-peptidic compounds, in addition to their utility as therapeutic compounds.

In one embodiment, a peptide can be characterized as a mimetic by containing one or more non-natural residues in place of a naturally occurring amino acid residue. Non-natural residues are known in the art. Particular non-limiting examples of non-natural residues useful as mimetics of natural amino acid residues are mimetics of aromatic amino acids include, for example, D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenyl-phenylalanine; and D- or L-2-indole(alkyl)alanines, where alkyl can be substituted or unsubstituted methyl, ethyl, propyl, hexyl, butyl, pentyl, isopropyl, iso-butyl, sec-isotyl, iso-pentyl, or a non-acidic amino acid. Aromatic rings of a non-natural amino acid that can be used in place a natural aromatic ring include, for example, thiazolyl, thiophenyl, pyrazolyl, benzimidazolyl, naphthyl, furanyl, pyrrolyl, and pyridyl aromatic rings. By way of illustration, Xaa₃ can be α-aminoisobutyric acid (Aib), aminobutyric acid (Abu), 2-aminopentanoic acid (Ape), 2-aminohexanoic acid (Ahx), or tert-leucine (Tle).

Cyclic peptides or cyclized residue side chains also decrease susceptibility of a peptide to proteolysis by exopeptidases or endopeptidases. Thus, certain embodiments embrace a peptidomimetic of the peptides disclosed herein, whereby one or more amino acid residue side chains are cyclized according to conventional methods.

Mimetics of acidic amino acids can be generated by substitution with non-carboxylate amino acids while maintaining a negative charge; (phosphono)alanine; and sulfated threonine. Carboxyl side groups (e.g., aspartyl or glutamyl) can also be selectively modified by reaction with carbodiimides (R′—N—C—N—R′) including, for example, 1-cyclohexyl-3(2-morpholinyl-(4-ethyl) carbodiimide or 1-ethyl-3(4-azonia-4,4-dimetholpentyl) carbodiimide. Aspartyl or glutamyl groups can also be converted to asparaginyl and glutaminyl groups by reaction with ammonium ions.

Lysine mimetics can be generated (and amino terminal residues can be altered) by reacting lysinyl with succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Methionine mimetics can be generated by reaction with methionine sulfoxide. Proline mimetics of include, for example, pipecolic acid, thiazolidine carboxylic acid, dehydroproline, 3- or 4-methylproline, and 3,3-dimethylproline.

One or more residues can also be replaced by an amino acid (or peptidomimetic residue) of the opposite chirality. Thus, any amino acid naturally occurring in the L-configuration (which can also be referred to as R or S, depending upon the structure of the chemical entity) can be replaced with the same amino acid or a mimetic, but of the opposite chirality, referred to as the D-amino acid, but which can additionally be referred to as the R- or S-form.

As will be appreciated by one skilled in the art, the peptidomimetics of the present invention can also include one or more of the modifications described herein for derivatized peptides, e.g., a label, one or more post-translational modifications, or cell-penetrating sequence.

Also included with the scope of the invention are peptides and peptidomimetics that are substantially identical to a sequence set forth herein, in particular SEQ ID NO:1 or SEQ ID NO:2. The term “substantially identical,” when used in reference to a peptide or peptidomimetic, means that the sequence has at least 75% or more identity to a reference sequence (e.g., 80%, 85%, 90%, 95%, 96%, 97%, 98%, 99%). The length of comparison sequences will generally be at least 6 amino acids, but typically more, at least 8 to 10, 8 to 13, 8 to 15, or 8 to 20 residues.

The peptides, derivatives and peptidomimetics can be produced and isolated using any method known in the art. Peptides can be synthesized, whole or in part, using chemical methods known in the art (see, e.g., Caruthers (1980) Nucleic Acids Res. Symp. Ser. 215-223; Horn (1980) Nucleic Acids Res. Symp. Ser. 225-232; and Banga (1995) Therapeutic Peptides and Proteins, Formulation, Processing and Delivery Systems, Technomic Publishing Co., Lancaster, Pa.). Peptide synthesis can be performed using various solid-phase techniques (see, e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the manufacturer's instructions.

Individual synthetic residues and peptides incorporating mimetics can be synthesized using a variety of procedures and methodologies known in the art (see, e.g., Organic Syntheses Collective Volumes, Gilman, et al. (Eds) John Wiley & Sons, Inc., NY). Peptides and peptide mimetics can also be synthesized using combinatorial methodologies. Techniques for generating peptide and peptidomimetic libraries are well-known, and include, for example, multipin, tea bag, and split-couple-mix techniques (see, for example, al-Obeidi (1998) Mol. Biotechnol. 9:205-223; Hruby (1997) Curr. Opin. Chem. Biol. 1:114-119; Ostergaard (1997)Mol. Divers. 3:17-27; and Ostresh (1996) Methods Enzymol. 267:220-234). Modified peptides can be further produced by chemical modification methods (see, for example, Belousov (1997) Nucleic Acids Res. 25:3440-3444; Frenkel (1995) Free Radic. Biol. Med 19:373-380; and Blommers (1994) Biochemistry 33:7886-7896).

In one embodiment, the peptide can be prepared in recombinant protein systems using polynucleotide sequences encoding the peptides. By way of illustration, a nucleic acid molecule encoding a peptide of the invention is introduced into a host cell, such as bacteria, yeast or mammalian cell, under conditions suitable for expression of the peptide, and the peptide is purified or isolated using methods known in the art. See, e.g., Deutscher et al. (1990) Guide to Protein Purification: Methods in Enzymology Vol. 182, Academic Press.

It is contemplated that the peptide conjugate of the invention as described herein can be used as lead compounds for the design and synthesis of compounds with improved efficacy, clearance, half-lives, and the like. One approach includes structure-activity relationship (SAR) analysis (e.g., NMR analysis) to facilitate the development of more efficacious agents. Agents identified in such SAR analysis or from agent libraries can then be screened for their ability to inhibit Aβ-induced synaptic depression mediated by PDZ-dependent recruitment of PTEN to dendritic spines.

For therapeutic applications, the peptide conjugate of the invention can be used as purified molecules (i.e., purified peptide conjugates, derivatives, or peptidomimetics), or in the case of peptide conjugates, be expressed from nucleic acids encoding the peptide moiety. Such nucleic acids can, if desired, be naked or be in a carrier suitable for passing through a cell membrane (e.g., DNA-liposome complex), contained in a vector (e.g., plasmid, retroviral vector, lentiviral, adenoviral or adeno-associated viral vectors and the like), or linked to inert beads or other heterologous domains (e.g., antibodies, biotin, streptavidin, lectins, etc.), or other appropriate compositions. Thus, both viral and non-viral means of nucleic acid delivery can be achieved and are contemplated. Desirably, a vector used in accordance with the invention provides all the necessary control sequences to facilitate expression of the peptide. Such expression control sequences can include but are not limited to promoter sequences, enhancer sequences, etc. Such expression control sequences, vectors and the like are well-known and routinely employed by those skilled in the art.

Based upon the findings that a peptide conjugate of the invention derived from the C-terminus of PTEN blocks Aβ-induced synaptic depression mediated by PDZ-dependent recruitment of PTEN and improves spatial learning in an animal model of Alzheimer's Disease, this invention provides in one embodiment, a method for mitigating or alleviating synaptic and cognitive deficits associated with a β-amyloidogenic disease using a peptide or mimetic described herein. As used herein, the terms “mitigating” or “alleviating” are meant to indicate delaying or even permanently delaying (i.e., preventing) development of synaptic and cognitive deficits and/or a reduction in the severity of synaptic and cognitive deficits that will, or are expected to, develop. The terms further include ameliorating existing symptoms or preventing additional symptoms. Therefore, the method of the invention encompasses applications to delay or arrest development of β-amyloidogenic disease in a subject at risk for such a disease. For instance, subjects with a genetic predisposition to Alzheimer's are suitable candidates for treatment according to the methods of the invention. The methods of the invention also encompass therapeutic treatment of a β-amyloidogenic disease in a subject diagnosed with such a disease. Advantageously, a peptide or mimetic inhibitor of the invention may reverse cognitive dysfunction and improve memory, such as spatial memory, and learning in a subject with Alzheimer's disease. Assays for determining the effectiveness of the peptide or mimetic of this invention include, but are not limited to, spatial learning tasks, memory tests and the like.

Diseases that may be treated by the method of the invention are β-amyloidogenic diseases. β-amyloidogenic diseases are characterized by the presence of Aβ plaques or deposits. For instance, Alzheimer's disease is characterized by mature senile plaques composed of Aβ in extracellular regions of the brain. β-Amyloidogenic diseases include, but are not limited to, Alzheimer's disease, Down's syndrome, mild cognitive impairment (MCI), cerebral amyloid angiopathy and hereditary cerebral hemorrhage with amyloidosis-Dutch type and -Icelandic type. In one embodiment of the invention, the β-amyloidogenic disease is Alzheimer's disease. Subjects suitable for treatment using the method of the invention are mammals, including humans.

In one embodiment, patients amenable to treatment include individuals at risk of disease but not showing symptoms, as well as patients presently showing symptoms. In the case of Alzheimer's disease, virtually anyone is at risk of suffering from Alzheimer's disease if he or she lives long enough. Therefore, the present methods can be administered prophylactically to the general population without any assessment of the risk of the subject patient. The methods of the invention are especially useful for individuals who do have a known genetic risk of Alzheimer's disease. Such individuals include those having relatives who have experienced this disease, and those whose risk is determined by analysis of genetic or biochemical markers. Genetic markers of risk toward Alzheimer's disease include mutations in the APP gene, particularly mutations at position 717 and positions 670 and 671 referred to as the Hardy and Swedish mutations respectively (see Hardy, TINS, supra). Other markers of risk are mutations in the presenilin genes, PS1 and PS2, and ApoE4, family history of AD, hypercholesterolemia or atherosclerosis. Individuals presently suffering from Alzheimer's disease can be recognized from characteristic dementia, as well as the presence of risk factors described above. In addition, a number of diagnostic tests are available for identifying individuals who have AD. These include measurement of CSF tau and Aβ42 levels. Elevated tau and decreased Aβ42 levels signify the presence of AD. Individuals suffering from Alzheimer's disease can also be diagnosed by MMSE or ADRDA criteria as discussed in the Examples section.

Thus, the present invention provides a method of preventing or treating a β-amyloidogenic disease comprising administering a pharmaceutically effective amount of a peptide conjugate of the invention, or a derivative or peptidomimetic thereof, to a subject in need thereof. In one embodiment, the β-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM). In one embodiment, the β-amyloidogenic disease is Alzheimer's disease.

The present invention further provides a method of preventing, mitigating, or alleviating synaptic or cognitive deficits associated with a β-amyloidogenic disease. In one embodiment, the β-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM). In one embodiment, the β-amyloidogenic disease is Alzheimer's disease.

In another aspect, the present invention provides a method of preventing or treating Alzheimer's disease comprising administering a pharmaceutically effective amount of a peptide conjugate of this invention, or a derivative or peptidomimetic thereof, to a subject in need thereof.

In another aspect, the present invention provides a method of treating symptoms of Alzheimer's disease comprising administering a pharmaceutically effective amount of a peptide conjugate of this invention, or a derivative or peptidomimetic thereof, to a subject in need thereof. In one embodiment, the symptoms in the method of the invention are mild cognitive impairment or age-associated memory loss. In one embodiment, such symptoms occur in some patients who have not yet developed or may never develop full Alzheimer's disease.

In one embodiment, this invention provides a method of enhancing cognitive function in healthy individuals. In one embodiment, this invention provides memory enhancement for healthy individuals. In one embodiment, this invention provides a method for the enhancement of cognitive function for subjects with age-related cognitive impairment. According to this aspect and in one embodiment, the medication can be used by students during study and/or while under examination, by employees at work, at home, at training sessions etc. Compositions comprising the peptide-conjugate of this invention can be administered to healthy individuals or to subjects with mild cognitive impairment in one embodiment. People with temporary memory loss may use compositions of the present invention as well. In other embodiments, subjects with severe memory loss, with permanent memory loss and with severe cognitive impairment may use compositions of this invention as a medication and can be the subjects for use of methods of this invention as described herein.

In one embodiment, the administering for the method of the invention is direct injection.

The peptide conjugate of the invention (including nucleic acids encoding the peptide moiety) can be formulated with a pharmaceutically acceptable carrier at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier, composition or vehicle, such as a liquid or solid filler, diluent, excipient, or solvent encapsulating material, is involved in carrying or transporting the subject agent from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In another aspect, the present invention provides a pharmaceutical composition comprising a peptide conjugate of the invention as described herein and a pharmaceutically acceptable adjuvants or carriers. In one embodiment, the pharmaceutical composition of the invention includes one or more additional pharmaceutically active agents or adjuvants conventionally used in the amelioration or treatment of 3-amyloidogenic diseases. For example, the inhibitor here can be used in combination with a cholinesterase inhibitor (e.g., donepezil HCl, rivastigmine, galantamine or tacrine), memantine, vitamin E, an antidepressant (e.g., citalopram, fluoxetine, paroxetine, sertraline or trazodone), an anxiolytic (e.g., lorazepam or oxazepam), or an antipsychotic (e.g., aripiprazole, clozapine, haloperidol, olanzapine or risperidone).

The pharmaceutical compositions of the invention that are useful in the methods of the invention may be prepared, packaged, or sold in formulations suitable for oral, rectal, vaginal, parenteral, topical, pulmonary, intranasal, buccal, ophthalmic, intrathecal, or another route of administration. Other contemplated formulations include nanoparticles and liposomal preparations containing the active ingredient. Controlled- or sustained-release formulations of a pharmaceutical composition of the invention may also be made using conventional technologies.

As used herein, “parenteral administration” of a composition includes any route of administration characterized by physical breaching of a tissue of a subject and administration of the pharmaceutical composition through the breach in the tissue. Parenteral administration thus includes, but is not limited to, administration of a pharmaceutical composition by direct injection of the composition, by application of the composition through a surgical incision, by application of the composition through a tissue-penetrating non-surgical wound, and the like. In particular, parenteral administration is contemplated to include, but is not limited to, intraventricular (into the brain's ventricles), subcutaneous, intraperitoneal, intramuscular, intrasternal injection, and kidney dialytic infusion techniques.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient, e.g., the peptide conjugate of the invention. The amount of the active ingredient is generally equal to the dosage of the active ingredient which would be administered to a subject or a convenient fraction of such a dosage such as, for example, one-half or one-third of such a dosage. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

A physician having ordinary skill in the art can readily determine and prescribe the effective amount of the pharmaceutical composition required based upon the administration of similar compounds or experimental determination. For example, the physician could start doses of an agent at levels lower than that required in order to achieve the desired therapeutic effect and gradually increase the dosage until the desired effect is achieved. This is considered to be within the skill of the artisan and one can review the existing literature on a specific agent or similar agents to determine optimal dosing.

The present invention is also directed to a kit to prepare and administer a composition containing a peptide conjugate of the invention or mimetic inhibitor that selectively blocks PDZ-dependent recruitment of PTEN into dendritic spines. The kit is useful for practicing the inventive method of treatment of β-amyloidogenic diseases such as Alzheimer's disease. The kit is an assemblage of materials or components, including at least one of the inventive compositions and a pharmaceutically acceptable carrier. Thus, in some embodiments, the kit contains a peptide derivative having the sequence Dodecanoyl-QHTQITKV (conjugate of peptide SEQ ID NO:1) or Dodecanoyl-QHSQITKV (conjugate of peptide SEQ ID NO:2) and a pharmaceutically acceptable carrier.

The exact nature of the components configured in the inventive kit depends on its intended purpose. For example, some embodiments are configured for the purpose of treating Alzheimer's disease. In one embodiment, the kit is configured particularly for the purpose of treating mammalian subjects. In another embodiment, the kit is configured particularly for the purpose of treating human subjects.

Instructions for use may be included in the kit. “Instructions for use” typically include a tangible expression describing the technique to be employed in using the components of the kit to affect a desired outcome, such as to monitor the improvement in cognitive function, memory and learning in a subject. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In one embodiment, peptide-conjugates of this invention are stable in human plasma for up to 6 hours. In one embodiment, peptide-conjugates of this invention are stable in human plasma for up to 24 hours. In one embodiment, at least 40% or at least 50% or at least 60% of the peptide-conjugates of this invention remain in human plasma after 4 h from the time of introducing to the human plasma. In one embodiment, at least 40% or at least 50% or at least 60% of the peptide-conjugates of this invention remain in human plasma after 6 h or after 24 h from the time of introducing to the human plasma. In one embodiment, between 40% and 80% of the peptide-conjugates of this invention remain in human plasma after 4 h from the time of introducing to the human plasma. In one embodiment, between 40% and 80% of the peptide-conjugates of this invention remain in human plasma after 6 h or after 24 h from the time of introducing to the human plasma. In one embodiment, after 4 h or after 6 h or after 8 h or after 10 h or after 12 h or after 18 h or after 24 h or after 48 h from the time of introducing to the human plasma, the percentage of the peptide-conjugate remaining in the human plasma ranges between 50% and 90% or between 40% and 95% or between 60% and 90% or between 60% and 80% or between 60% and 95% or between 50% and 80%. In one embodiment, after 24 h or after 48 h from the time of introducing to the human plasma, the percentage of the peptide-conjugate remaining in the human plasma ranges between 10% and 40% or between 20% and 50% or between 5% and 30% or between 10% and 30%. In one embodiment, after 24 h or after 48 h from the time of introducing to the human plasma, the percentage of the peptide-conjugate remaining in the human plasma is at least 2.5% or at least 5% or at least 10% or at least 20% or at least 30% or at least 40% or at least 50%.

In one embodiment, cell metabolic activity after exposure to the peptide conjugates at various concentrations for at least 4 h is at least 50% of the cell activity under no exposure to the peptide-conjugates of this invention. In one embodiment, cell metabolic activity after exposure to the peptide conjugates at various concentrations for at least 4 h is at least 40% or at least 50% or at least 60% or at least 70% or at least 80% of the cell activity under no exposure to the peptide-conjugates of this invention. In one embodiment, the various concentration range is 0.001M to 0.1 M. In one embodiment, the various concentration range is 0.01M to 0.1 M. In one embodiment, the various concentration range is 0.0001 M to 1 M.

In one embodiment, cell metabolic activity after exposure to the peptide conjugates at various concentrations for at least 4 h ranges between 40% and 60% of the cell activity under no exposure to the peptide-conjugates of this invention. In one embodiment, cell metabolic activity after exposure to the peptide conjugates at various concentrations for at least 4 h ranges between 50% and 90% or between 40% and 95% or between 60% and 90% or between 40% and 90% or between 60% and 80% or between 60% and 95% or between 50% and 80% or between 70% and 95% or between 80% and 95% of the cell activity under no exposure to the peptide-conjugates of this invention.

In one embodiment, cell metabolic activity after exposure to the peptide conjugates at various concentrations for at least 24 h is at least 80% of the cell activity under no exposure to the peptide-conjugates of this invention. In one embodiment, cell metabolic activity after exposure to the peptide conjugates at various concentrations for at least 24 h is at least 90% or at least 95% or at least 60% or at least 70% or at least 75% or at least 99% of the cell activity under no exposure to the peptide-conjugates of this invention.

In one embodiment, cell metabolic activity after exposure to the peptide conjugates at various concentrations for at least 24 h ranges between 75% and 100% of the cell activity under no exposure to the peptide-conjugates of this invention. In one embodiment, cell metabolic activity after exposure to the peptide conjugates at various concentrations for at least 24 h ranges between 75% and 95% or between 90% and 100% or between 95% and 100% or between 80% and 99% or between 80% and 100% or between 70% and 95% or between 50% and 100% or between 50% and 95% or between 50% and 99% or between 75% and 99% or between 75% and 100% or between 70% and 100% or between 80% and 99% or between 85% and 100% or between 70% and 95% or between 50% and 100% or between 50% and 95% or between 50% and 99% of the cell activity under no exposure to the peptide-conjugates of this invention.

In one embodiment, cell metabolic activity after exposure to the peptide conjugates at various concentrations for at least 24 h remains 100% of the cell activity under no exposure to the peptide-conjugates of this invention.

In one embodiment, the permeability of the peptide-conjugates of this invention across human BBB is evaluated by determination of the apparent permeability coefficient (Papp). According to this aspect and in one embodiment, the apparent permeability coefficient for peptide-conjugates of this invention is 6.3±1.8×10⁻⁶ cm/s.

In one embodiment, the apparent permeability coefficient for peptide-conjugates of this invention is at least 6.3×10⁻⁶ cm/s. In one embodiment, the apparent permeability coefficient for peptide-conjugates of this invention is at least 6.0×10⁻⁶ cm/s. In one embodiment, the apparent permeability coefficient for peptide-conjugates of this invention is at least 3.8×10⁻⁶ cm/s or at least 3.9×10⁻⁶ cm/s or at least 4.0×10⁻⁶ cm/s or at least 5.0×10⁻⁶ cm/s or at least 5.5×10⁻⁶ cm/s or at least 6.0×10⁻⁶ cm/s or at least 6.5×10⁻⁶ cm/s or at least 7.0×10⁻⁶ cm/s.

In one embodiment, the apparent permeability coefficient for peptide-conjugates of this invention ranges between 5.0×10⁻⁶ cm/s and 7.0×10⁻⁶ cm/s. In one embodiment, the apparent permeability coefficient for peptide-conjugates of this invention ranges between 3.8×10⁻⁶ cm/s and 7.0×10⁻⁶ cm/s or between 3.9×10⁻⁶ cm/s and 7.0×10⁻⁶ cm/s or between 4.0×10⁻⁶ cm/s and 7.0×10⁻⁶ cm/s or between 5.0×10⁻⁶ cm/s and 6.0×10⁻⁶ cm/s or between 5.0×10⁻⁶ cm/s and 6.5×10⁻⁶ cm/s or between 4.0×10⁻⁶ cm/s and 6.0×10⁻⁶ cm/s or between 4.0×10⁻⁶ cm/s and 6.5×10⁻⁶ cm/s.

In one embodiment, the human BBB is an in vitro 2D human BBB, results for which are shown in table 5 herein below.

PTEN 15 phosphatase and tensin homolog. PDZ is an initialism combining the first letters of the first three proteins discovered to share the domain—post synaptic density protein (PSD95), drosophila disc large tumor suppressor (Dlg1), and zonula occludens-1 protein (zo-1). SEM is standard error of mean in certain embodiments. BBB is blood brain barrier. MTT is 3-(4,5-dimethylthiazol-2-yl)-2,5-diphenyltetrazolium bromide.

In some embodiment, peptide conjugates of this invention are referred to in short as ‘conjugates’ or as ‘peptides’. In one embodiment, ‘residue’ means amino acid residue.

In one embodiment, this invention provides a peptide conjugate N-Dodecanoyl-QHTQITKV (conjugate of peptide SEQ ID NO:1), derivative or peptidomimetic thereof. In one embodiment, this invention provides a peptide conjugate N-Dodecanoyl-QHSQITKV (conjugate of peptide SEQ ID NO:2), derivative or peptidomimetic thereof.

In one embodiment, this invention provides a method of preventing or treating a β-amyloidogenic disease, the method comprising administering a pharmaceutically effective amount of the peptide conjugate SEQ ID NO:1 or the peptide conjugate SEQ ID NO:2 described herein above, to a subject in need thereof. In one embodiment, the 3-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM).

In one embodiment, this invention provides a method of preventing, mitigating, or alleviating synaptic or cognitive deficits associated with a β-amyloidogenic disease, the method comprising administering a pharmaceutically effective amount of the peptide conjugate SEQ ID NO:1 or the peptide conjugate SEQ ID NO:2 as described herein above, to a subject in need thereof. In one embodiment, the β-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM).

In one embodiment, this invention provides a method of preventing or treating Alzheimer's disease, the method comprising administering a pharmaceutically effective amount of the peptide conjugates described herein above, to a subject in need thereof. In one embodiment, the peptide conjugates are selected from peptide conjugate SEQ ID NO:1 or peptide conjugate SEQ ID NO:2 as described herein above.

In one embodiment, this invention provides a method of treating symptoms of Alzheimer's disease comprising administering a pharmaceutically effective amount of peptide conjugate SEQ ID NO:1 or of peptide conjugate SEQ ID NO:2 as described herein above, to a subject in need thereof. In one embodiment, the symptoms are mild cognitive impairment or age-associated memory loss.

In one embodiment, this invention provides a method of improving age-related memory impairment and enhancing cognitive function in healthy individuals, the method comprising administering a pharmaceutically effective amount of the peptide conjugates described herein above, to a subject in need thereof.

In one embodiment, administering is conducted by injection.

In one embodiment, this invention provides a composition comprising the peptide conjugates of this invention and a pharmaceutically acceptable carrier. In one embodiment, this invention provides a composition comprising a peptide conjugate N-Dodecanoyl-QHTQITKV (conjugate of peptide SEQ ID NO:1), derivative or peptidomimetic thereof and a pharmaceutically acceptable carrier. In one embodiment, this invention provides a composition comprising a peptide conjugate N-Dodecanoyl-QHSQITKV (conjugate of peptide SEQ ID NO:2), derivative or peptidomimetic thereof and a pharmaceutically acceptable carrier.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

### Example 1

### Materials and Methods

Animals

Wild-type littermates were used as controls in each of the experiments involving transgenic mice. At weaning, the mice were genotyped from tail biopsies by means of polymerase chain reaction. The following mouse lines were used in this study:

APP/PS1 Mice.

Double transgenic (B6-Cg-Tg(APPswe, PSEN1dE9)85Dbo-J) mice were used for behavioral and biochemical experiments (male, age, 5 months at the end of the experiment). PCR-genotyping was carried out with three specific sense primers for PS1 (5′-CAGGTGGTGGAGCAAGATG; SEQ ID NO:24), APP (5′-CCGAGATCTCTGAAGTGAAGATGGATG; SEQ ID NO:25), and PrP (5′-CCTCTTTGTGACTATGTGGACTGATGTCGG; SEQ ID NO:26), and one common antisense primer matching the sequence within PrP (5′-GTGGATACCCCCTCCCCCAGCCTAGACC; SEQ ID NO:27) (Lesuisse, et al. (2001)Hum. Mol. Genet. 10:2525-2537). The PCR genotyping results were confirmed by histology using Thioflavin-S stain and by measurements of Aβ monomers (42 and 40) with ELISA.

Peptide Synthesis

All peptides were prepared manually using Fmoc-based solid-phase synthesis protocols with HCTU as the coupling agent (Hood, et al. (2008) J. Pept. Sci. 14:97-101). Fluorescently-labeled peptides were prepared by adding a Fmoc-Lys residue (with Mtt side chain protecting group) to the N-terminus of the peptide while on resin, selectively removing the Mtt protecting group, and covalently attaching fluorescein through reaction with 5-FITC (fluorescein-5-isothiocyanate). All peptides were purified using semi-preparative, reverse-phase HPLC (RP-HPLC) using a C18 column with water-methanol mobile phase gradient, followed by lyophilization to yield white solids. Molecular mass of each purified peptide was confirmed by LCMS analysis (Shimadzu LCMS-2020).

Dodecanoyl-QHSQITKV (conjugate of peptide SEQ ID NO:2) and Dodecanoyl-QHTQITKV (conjugate of peptide SEQ ID NO: 1) were synthesized. Compound composition has been verified by HPLC and by mass spectrometry tests.

For Dodecanoyl-QHSQITKV (conjugate of peptide SEQ ID NO:2) (Dodecanoyl conjugated to the N-terminus), analysis results were as follows: theoretical MW of peptide 1122.40, MW (MW+H⁺) measured by MS was 1123.85. MW measured by MS was 1122.85. Purity 99.45%. Format: Lyophilized trifluoroacetate salt.

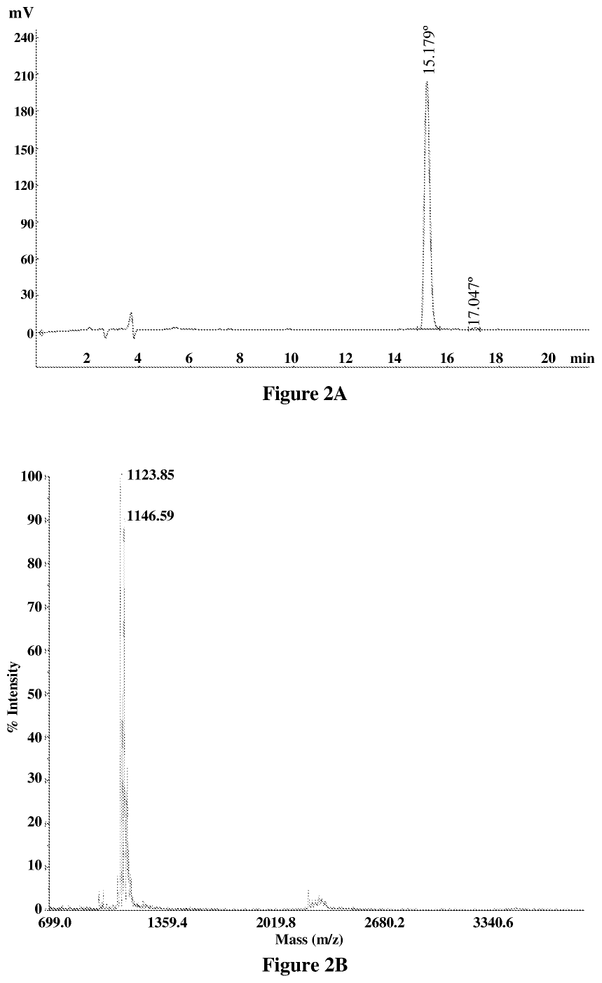

HPLC results are shown in FIG. 2A. HPLC column (250×4.6 mm I.D.) C18. Detection wavelength 220 nm. Gradient 30-52% B in 22 min. Buffer A 0.05% TFA+2% CH₃CN. Buffer B 0.05% TFA+90% CH₃CN. Peak results:

TABLE 2

| Rank | Time | Conc. | Area | Height |
|---|---|---|---|---|
| 1 | 15.179 | 99.45 | 2770673 | 201437 |
| 2 | 17.047 | 0.5478 | 15262 | 1101 |
| Total | | 100 | 2785953 | 202538 |

Mass spectrometry results are shown in FIG. 2B. Method MALDI-TOF, main peak 1123.85; MW [M+H⁺] 1123.85; MW: 1122.85; Theoretical MW: 1122.40. Match: Approved. Z=1

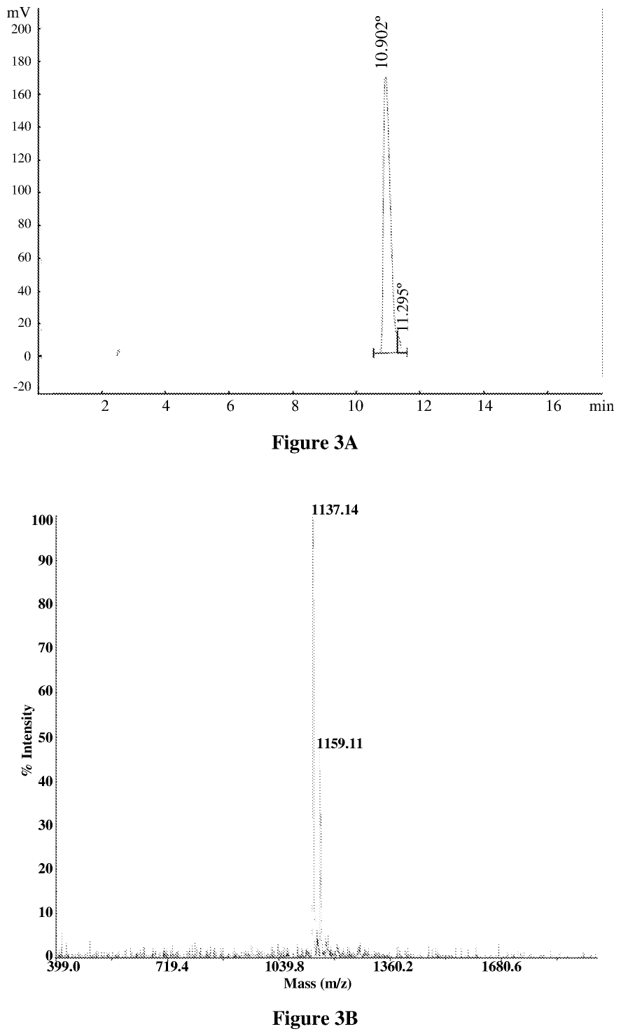

For Dodecanoyl-QHTQITKV (conjugate of peptide SEQ ID NO:1), HPLC results are shown in FIG. 3A and mass spectrometry results are shown in FIG. 3B.

For Dodecanoyl-QHTQITKV (conjugate of peptide SEQ ID NO:1) (Lauric acid (Dodecanoyl) conjugated to the N-terminus), analysis results were as follows: theoretical MW of peptide 1136.46, MW (MW+H⁺) measured by MS was 1137.14. MW measured by MS was 1136.14. Match: Approved. Purity 98.14%. Format: Lyophilized trifluoroacetate salt.

HPLC results are shown in FIG. 3A. HPLC column (250×4.6 mm I.D.) C18. Detection wavelength 220 nm. Gradient 35-53% B in 18 min. Buffer A 0.05% TFA+2% CH₃CN. Buffer B 0.05% TFA+90% CH₃CN. Peak results:

TABLE 3

| Rank | Time | Conc. | Area | Height |
|---|---|---|---|---|
| 1 | 10.902 | 98.14 | 2357693 | 169186 |
| 2 | 11.295 | 1.864 | 44783 | 10484 |
| Total | | 100 | 2402476 | 179670 |

Mass spectrometry results are shown in FIG. 3B. Method MALDI-TOF, main peak 1137.14; MW [M+H⁺] 1137.14; MW: 1136.14; Theoretical MW: 1136.46. Match: Approved. Z=1.

Behavioral Tests

Use of peptides in vivo with osmotic pumps. APP/PS1 (a mouse model of Alzheimer's disease) and WT mice were anesthetized with isofluorane, and i.c.v. delivery cannulas (brain alzet kit III) were implanted with a stereotaxic frame at the following coordinates according to the bregma: AP, −0.5 mm; ML, 1 mm; and DV, −2.2 mm. Osmotic minipumps (Alzet; model #1004) were filled with the peptides (Dodecanoyl-QHSQITKV (conjugate of peptide SEQ ID 2) or Dodecanoyl-QHTQITKV (conjugate of peptide SEQ ID 1), 10 μM peptide in 2 mM in cyclodextrin) or with vehicle (cyclodextrin), and equilibrated in 0.9% NaCl at 37° C. for 48 hr. They were attached to the i.c.v. cannula tubing and subcutaneously implanted at the back. After 21 days, behavioral testing was started. Animal manipulation and data analysis were carried out blind with respect to genotype and treatment.

### Example 2

### Effect of the Peptide-Conjugate on Cognitive Function-Barnes Maze

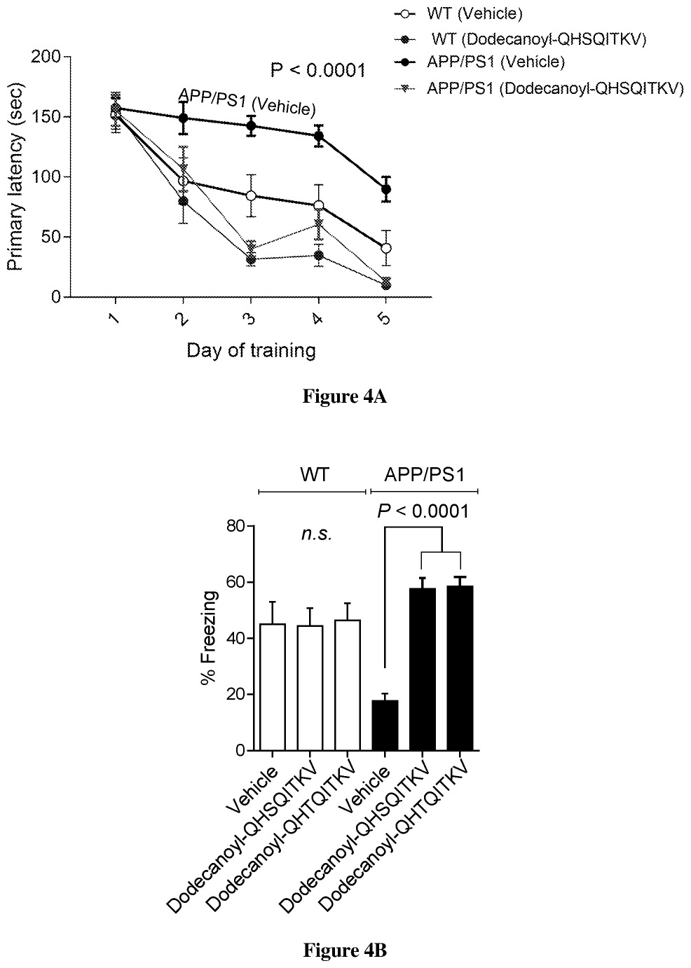

Barnes Maze. The Barnes circular maze (Barnes, 1979) was employed to assess spatial reference memory by training an animal to locate a hidden escape tunnel located directly beneath one of the holes at the perimeter of a large circular platform, which was brightly lit to provide a low-level aversive stimulus. Mice learn the location of the escape hole with the help of spatial reference points. This maze is an alternative to the Morris water maze (Morris, 1984) because it is considered to be less anxiogenic given that it does not involve swimming. The Barnes maze consists of a flat, circular disk with twenty holes around its perimeter that permit the animal to exit the maze into an escape box. Only one hole is open (the escape hole), which has a cage underneath with clean bedding (the escape box). The maze was divided into four quadrants of equal size, placing the escape hole in the middle of one of the quadrants equidistant from the sidewalls. The testing room contained numerous maze cues. The behavior of the animal was monitored with a video camera mounted on the ceiling above the center of the maze, computerized by a tracking system. Each training session includes four trials of 5 minutes each with 30 s intertrial intervals between trials 1-2 and 3-4, and a 30-minute intertrial interval between trials 2-3. The starting point was the middle of the maze, where the animals were placed for 30 s before each trial. A training session ended when the mouse entered the escape hole or when 5 min had elapsed, whichever came first. If the mouse did not find the escape hole within the 5 minutes of the trial, the experimenter gently guided the animal to the escape hole. In this case, the escape latency was recorded as 300 s and the mouse was then allowed to remain in the cage for 30 s. The parameter analyzed was the primary latency to find the escape hole (how long was required for the mouse to find the escape hole of the first time). As can be seen in FIG. 4A, the learning curve of APP/PS1 (treated with vehicle) was slow compared to WT mice, implying worse learning capabilities. Nevertheless, APP/PS1 mice treated with the Dodecanoyl-QHSQITKV (conjugate of peptide SEQ ID 2) peptide presented a learning curve that was similar to the learning curve of WT mice.

FIG. 4A shows performance in the Barnes maze. WT and APP/PS1 mice were trained in the Barnes maze (4 trials per day for five days). The latency to reach the escape box was recorded. As can be seen, Dodecanoyl-QHSQITKV (conjugate of peptide SEQ ID 2) improves learning in this task, in APP/PS1 mice, generally showing poor performance in this task (see inverted triangle graph vs. upper filled-circles graph). Improved learning is also shown for WT treated with Dodecanoyl-QHSQITKV (conjugate of peptide SEQ ID 2) (see lower curve of filled circles (WT Dodecanoyl-QHSQITKV (conjugate of peptide SEQ ID 2)) vs. central curve of unfilled circles (WT vehicle).

### Example 3

### Effect of the Peptide-Conjugate on Cognitive Function-Fear Conditioning

Fear conditioning. In this test, mice form an association between a certain context (an experimental cage/tone) and an aversive event (a foot shock) that takes place in that context. When placed back into the context, mice exhibit a range of conditioned fear responses, including immobility (freezing). Training and testing took place in a rodent observation cage (30×37×25 cm) that was placed in a sound attenuating chamber. In the training (conditioning), the mouse was exposed to the conditioning context (180 sec) followed by a tone (CS, 20 sec, 2 kHz, 85 dB). After termination of the tone, a foot shock (US, 0.75 mA, 2 sec) was delivered through a stainless-steel grid floor. Mice received three foot shocks with an intertrial interval of 60 s. The mouse was removed from the fear conditioning box 30 sec after shock termination and returned to their home cages. Testing: In the contextual fear conditioning version, mice were placed back into the original training context for 8 min, during which no foot shock was delivered. In the auditory-cued fear-conditioning version, animals were placed into a novel context (same cages, but with different walls, floor, and background odor), and after a 3 min baseline period, they were continuously re-exposed to the tone (same characteristics as at conditioning) for 5 min, but in the absence of shocks. The animals' behavior was scored by an observer blind to the treatment conditions. Using a time-sampling procedure every 2 s, each mouse was scored blindly as either freezing or active at the instant the sample was taken. Freezing was defined as behavioral immobility except for movement needed for respiration.

To test whether Dodecanoyl-QHSQITKV (conjugate of peptide SEQ ID 2) or Dodecanoyl-QHTQITKV (conjugate of peptide SEQ ID 1) treatment can prevent cognitive impairment in the Alzheimer's mouse model, Dodecanoyl-QHSQITKV (conjugate of peptide SEQ ID 2), Dodecanoyl-QHTQITKV (conjugate of peptide SEQ ID 1) or vehicle were infused over a period of 3-4 weeks into the brain ventricles of 4-month-old APP/PS1 mice and their wild-type (WT) littermates using osmotic minipumps. Mice were then tested on contextual fear conditioning. As shown in FIG. 4B both peptides improved the performance of APP/PS1 mice in the fear conditioning test.

FIG. 4B shows the results of the contextual fear conditioning. The graph shows freezing time (in percentage) in WT and APP/PS1 mice treated with vehicle or with the peptides (Dodecanoyl-QHSQITKV (conjugate of peptide SEQ ID 2) or Dodecanoyl-QHTQITKV (conjugate of peptide SEQ ID 1)). As can be seen, both peptides enhance freezing behavior in APP/PS1 mice, thus, contextual fear memory in enhanced.

### Example 4

### Fear Conditioning-Comparative Results

The performance of myristoyl conjugate was compared with the performance of the dodecanoyl conjugate in a fear conditioning test. It was shown that dodecanoyl peptides (dodecanoyl-QHSQITKV (conjugate of peptide SEQ ID 2) or dodecanoyl-QHTQITKV (conjugate of peptide SEQ ID 1)) enhance freezing behavior in APP/PS1 mice more than the Myristoyl peptide conjugate (myristoyl-QHSQITKV (conjugate of peptide SEQ ID 2)). The increase in freezing of the dodecanoyl conjugate compared to myristoyl-QHSQITKV (conjugate of peptide SEQ ID 2) is by 36% (for Dodecanoyl-QHTQITKV (conjugate of peptide SEQ ID 1) vs. myristoyl conjugate) and by 37% (for Dodecanoyl-QHSQITKV (conjugate of peptide SEQ ID 2) vs. the myristoyl conjugate).

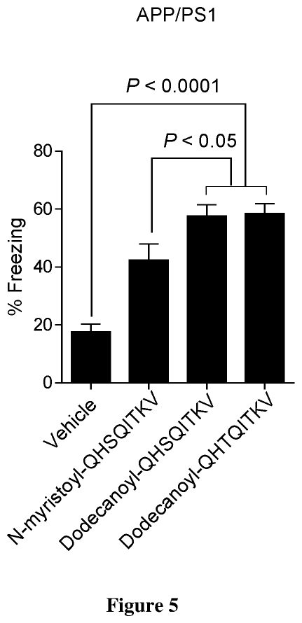

FIG. 5 shows the results of the contextual fear conditioning comparing the myristoyl derivative with the Dodecanoyl derivatives. The graph shows freezing time (in percentage) in APP/PS1 mice treated with vehicle or with the peptides (myristoyl-QHSQITKV (conjugate of peptide SEQ ID 2), dodecanoyl-QHSQITKV (conjugate of peptide SEQ ID 2) or Dodecanoyl-QHTQITKV (conjugate of peptide SEQ ID 1)). As shown in the figure, both dodecanoyl peptides enhance freezing behavior in APP/PS1 mice more than the myristoyl peptide.

### Example 5

### Stability

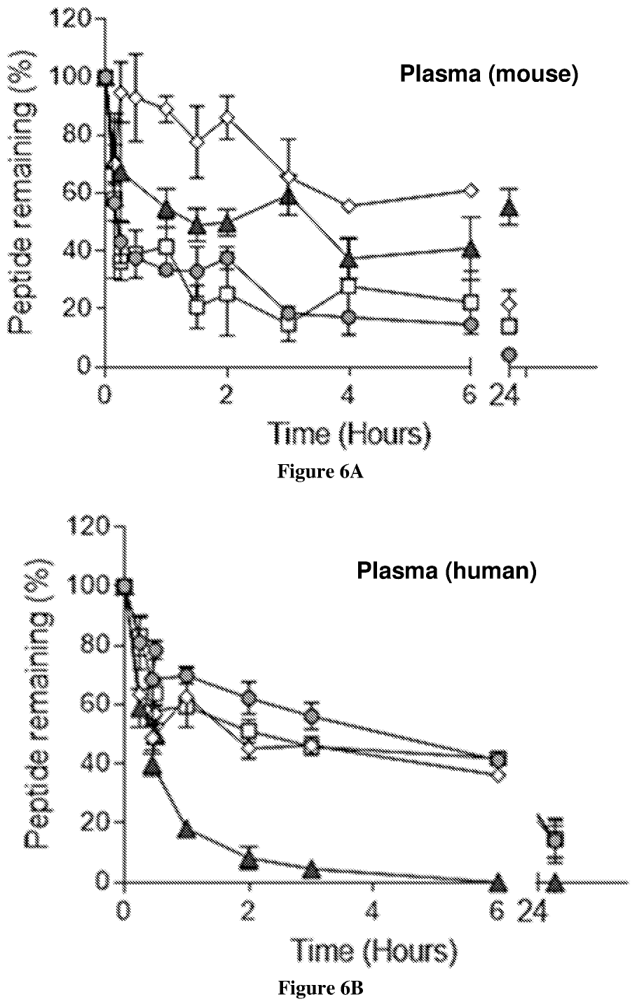

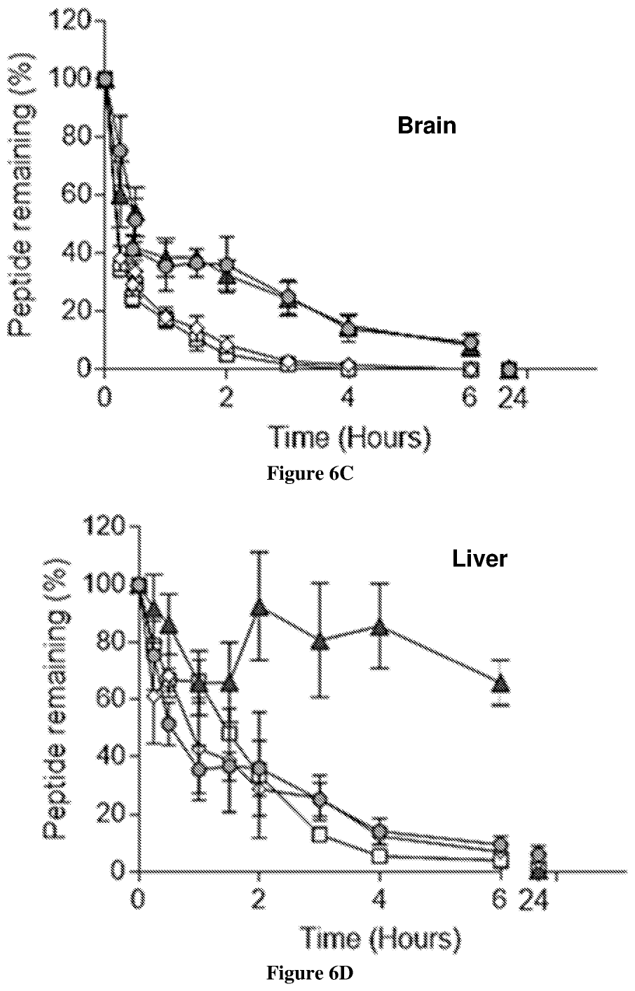

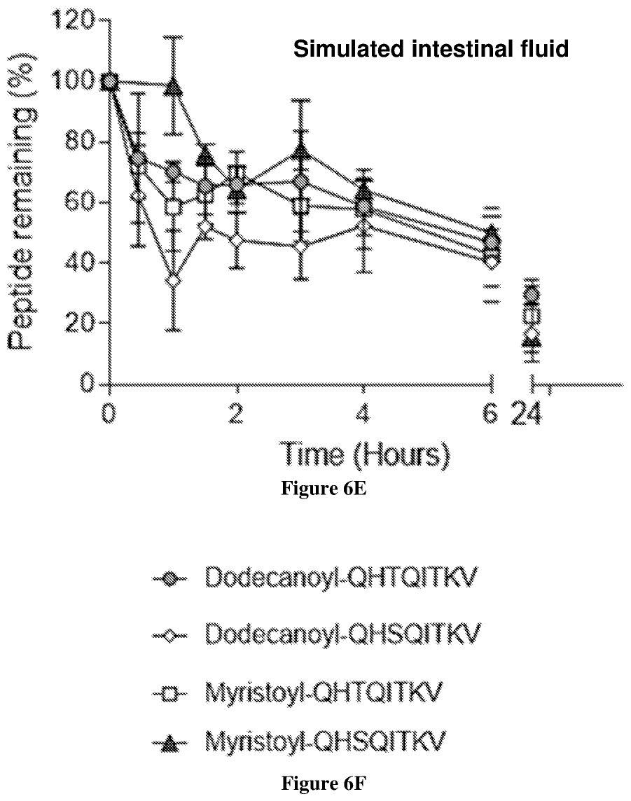

Plasma stability. Our data indicate that the lipidized peptides tested are generally more stable in human plasma compared to mouse plasma. Myristoyl-QHSQITKV and Dodecanoyl-QHSQITKV are more plasma stable in mouse plasma exhibiting longer half-lives and ˜50% of peptide remaining after 2 hours of incubation. Based on the higher lipophilicity of myristoylated peptides, dodecanoyl peptides are expected to be less protein bound and a higher percentage of the peptide will be available for permeation across biological barriers such as the BBB (only free fraction is available for transport). Thus, Dodecanoyl-QHSQITKV is a likely candidate for further development. Dodecanoyl-QHSQITKV demonstrated a plasma half-life of 5.9 hours in mice and two-phase decay in human plasma involving a quick phase with a half-life of 0.1 h followed by a longer half-life of 6.1 h (FIG. 6, Table 4).

Liver stability (mouse). Clearance of peptide therapeutics is highly dependent on the high metabolism occurring by liver enzymes able to degrade within minutes the total dose of peptide reaching the bloodstream after intravenous, other parenteral and non-invasive (oral and nasal routes). Peptides with good plasma and liver stability are more likely to remain in circulation and thus elicit higher levels if peptide is brain permeable across the BBB via invasive and non-invasive routes. Similarly to plasma, mouse peptides demonstrated higher liver stability with N-Dodecanoyl-QHSQITKV demonstrating a half-life of ˜1 h and N-Myristoyl-QHSQITKV of 1.4 h (Table 1).

Brain stability (mouse). Brain stability data shows similarities to the liver data and Dodecanoyl-QHSQITKV demonstrated a half-life of 0.20 h compared to Myristoyl-QHSQITKV with a half-life of 0.26 h. Our data indicate that Myristoyl-QHSQITKV and Dodecanoyl-QHTQITKV are more brain stable exhibiting longer half-lives (FIG. 6).

Simulated intestinal fluids. The first hurdle that a peptide faces upon oral delivery is the high salt and acidic environment of the stomach that can denature the therapeutic peptide as well as the action of pepsin. Enteric coating can overcome this first hurdle easily with currently available industrial processes. Thus, we assessed the stability of the lipidized peptides in simulated intestinal fluids from non-fasted animals i.e. soluble fraction of digestive enzymes present in the upper gastrointestinal (and the major absorptive site for peptides). This soluble fraction contains pancreatic proteases such as trypsin and chymotrypsin, carboxypeptidases and aminopeptidases able to hydrolyse peptides down to di- or tripeptides. Myristoylated peptides are more stable in simulated intestinal enzymes (FIG. 6) and if oral delivery is considered Myristoyl-QHSQITKV may have an advantage in at least eliciting the higher oral bioavailability and thus plasma levels. However, permeability across the BBB needs to be taken into consideration. Table 4 below shows a summary of half-lives for the peptides.

TABLE 4

| | | | |
|---|---|---|---|
| Equation fitted | k (h⁻¹) | t_{1/2} (h)/t_{1/2} (min) | r² |

Plasma (Mouse)

| | | | | |
|---|---|---|---|---|
| Dodecanoyl-QHTQITKV | One phase decay, Least | 4.595 | 0.1508/9.048 | 0.8294 |
| | square fit | | | |
| Dodecanoyl- QHSQITKV | One phase decay, Least | 0.1182 | 5.864/351.840 | 0.5959 |
| | square fit | | | |
| Myristoyl-QHTQITKV | One phase decay, Least | 6.172 | 0.1123/6.738 | 0.57 |
| | square fit | | | |
| Myristoyl-QHSQITKV | One phase decay, Least | 0.7972 | 0.8694/52.164 | 0.1714 |
| | square fit | | | |

Plasma (Human)

| | | | | |
|---|---|---|---|---|
| Dodecanoyl-QHTQITKV | One phase decay, Least | 0.2102 | 3.298/197.88 | 0.8742 |
| | square fit | | | |
| Dodecanoyl- QHSQITKV | One phase decay, Least | 2.751 | 0.252/15.12 | 0.7205 |
| | square fit | | | |
| | Two phase decay | 6.876 (fast), | 0.1008 (fast) & 7.563 | 0.8962 |
| | | 0.09165 | (slow)/6.05 (fast) & | |
| | | (slow) | 453.78 (slow) | |
| Myristoyl-QHTQITKV | One phase decay, Least | 1.604 | 0.4321/25.926 | 0.57 |
| | square fit | | | |
| Myristoyl-QHSQITKV | One phase decay, | 0.3116 | 2.224/133.44 | 0.7899 |
| | Least square fit | | | |

Brain

| | | | | |
|---|---|---|---|---|
| Dodecanoyl-QHTQITKV | One phase decay, Least | 1.901 | 0.3646/21.876 | 0.8852 |
| | square fit | | | |
| Dodecanoyl- QHSQITKV | One phase decay, Least | 3.481 | 0.1991/11.946 | 0.8395 |
| | square fit | | | |
| Myristoyl-QHTQITKV | One phase decay, Least | 4.204 | 0.1649/9.894 | 0.6133 |
| | square fit | | | |
| Myristoyl-QHSQITKV | One phase decay, Least | 2.714 | 0.2554/ 15.324 | 0.7841 |
| | square fit | | | |

Liver

| | | | | |
|---|---|---|---|---|
| Dodecanoyl-QHTQITKV | One phase decay, Least | 1.146 | 0.6046/36.276 | 0.7874 |
| | square fit | | | |
| Dodecanoyl- QHSQITKV | One phase decay, Least | 0.7278 | 0.9524/57.144 | 0.7528 |
| | square fit | | | |
| Myristoyl-QHTQITKV | One phase decay, Least | 0.03747 | 18.5/1110 | 0.4879 |
| | square fit | | | |
| | Two phase decay | 3.051 (fast), | 0.2272 (fast) & 1510 | 0.5364 |
| | | 0.00046 | (slow)/13.632 (fast) & | |
| | | (slow) | 90,600 (slow) | |
| Myristoyl-QHSQITKV | One phase decay, | 0.5092 | 1.361/81.66 | 0.8542 |
| | Least square fit | | | |

Simulated intestinal fluid

| | | | | |
|---|---|---|---|---|
| Dodecanoyl-QHTQITKV | One phase decay, Least | 0.2337 | 2.966/177.96 | 0.2216 |
| | square fit | | | |
| Dodecanoyl- QHSQITKV | One phase decay, Least | 2.340 | 0.2962/17.772 | 0.4753 |
| | square fit | | | |
| Myristoyl-QHTQITKV | One phase decay, Least | 0.2490 | Could not be | 0.1183 |
| | square fit | | calculated due to | |
| | | | fitting (deviation) | |
| Myristoyl-QHSQITKV | One phase decay, Least | 0.1534 | 4.517/271.02 | 0.7422 |
| | square fit | | | |

### Example 6

### Permeability Across an In Vitro 2D Human BBB Model

The in vitro permeability across a human 2D blood-brain Transwell model (hCMEC/D3 and human astrocytes SC-1800) was tested and enabled the calculation of the apparent permeability coefficient (Papp) for all peptides. Dodecanoyl peptides demonstrated almost double permeability across the BBB compared to myristoylated peptides with Dodecanoyl-QHSQITKV demonstrating the highest permeability 6.3±1.8×10⁻⁶ cm/s. Papp values calculated for controls (FITC-Dextran and diazepam) match previous reports. Papp for Dodecanoyl-QHSQITKV are in the same range with previous reported values for brain permeable peptides e.g. Angiopep-2 Papp (cm/s): 8.69±1.53×10⁻⁶ and is likely to yield to levels of 0.1-0.2% of an intravenously injected dose (i.e. plasma levels) crossing the BBB.

Table 5 below shows permeability of compounds across the in vitro 2D human BBB model in 2 separate experiments (n=3/experiment).

TABLE 5

| | Papp_{0-4 h} (×10⁻⁶ cm s⁻¹) | Papp_{0-4 h} (×10⁻⁶ cm s⁻¹) |
|---|---|---|
| | experiment 1 | experiment 2 |
| Dodecanoyl-QHTQITKV | | 5.5731 ± 0.2891 |
| Dodecanoyl- QHSQITKV | 6.2762 ± 1.8480 | |
| Myristoyl-QHTQITKV | | 3.7670 ± 0.3863 |
| Myristoyl-QHSQITKV | | 2.6345 ± 0.0786 |
| FITC-Dextran (3-5 kDa) | 6.9132 ± 0.3047 | |
| Diazepam | 19.0742 ± 0.5757 | 18.9031 ± 0.8643 |

### Example 7

### Toxicity: Cell Metabolic Activity Assays in hCMEC/D3 Cells

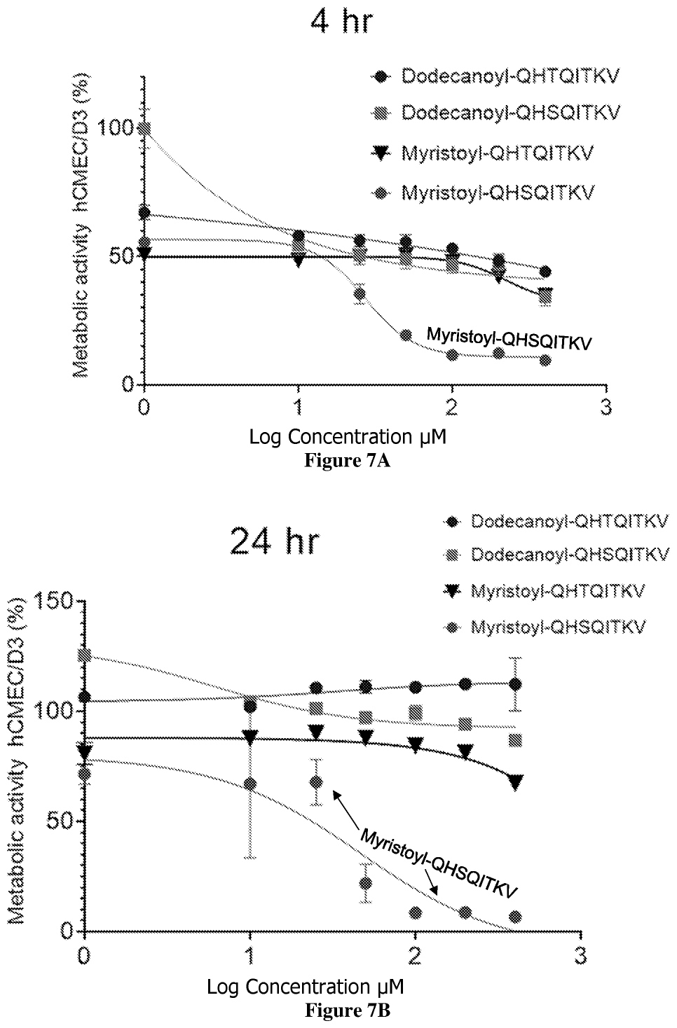

Cell metabolic activity was calculated in Human cerebral microvascular endothelial cells (hCMEC/D3) after exposure to the peptides at various concentrations for 4 or 24 hours (n=3, FIG. 7). We subtracted the values at 690 nm from 570 nm to remove background, and dividing the values by the control to express as a percentage (%) of the control (0.5% DMSO):

$$Cell Metabolic Activity ( \% ) = \frac{( A b {s}_{570 nm Sample} - Ab {s}_{690 nm Sample} ) \times 100}{( A b {s}_{570 nm Control} - Ab {s}_{690 nm Control} )}$$

At the specified time points, the MTT solution (20 μL at 5 mg mL⁻¹ solution in PBS) were added to each well and cells were incubated for 4 hours at 37° C. Subsequently, DMSO (100 μL) was added to dissolve the formazan crystals and absorbance was measured at 570 and 690 nm using Multiskan Go microplate spectrophotometer and data were analysed using the SkanIt software (Thermo Scientific, Paisley, UK). Cell metabolic activity was calculated by subtracting the values at 690 nm from 570 nm to remove background, and dividing the values by the control to express as a percentage (%) of the control (0.5% DMSO):

The MTT assay demonstrate some acute toxicity at high peptide concentrations in hCMEC/D3 (4 hours) but cells were able to recover at 24 hours with more than 80% of the cells remaining metabolically active at 200 μM (concentration used for BBB permeability assays) for all peptides except Myristoyl-QHSQITKV that was the only peptide where cells did not recover at 24 hours (see FIG. 7).

### Example 8

### Self-Assembly and Morphological Examination

Peptides were able to aggregate in aqueous media and interact with Thioflavin T (ThT). ThT can be immobilized in fibrils/aggregates resulting in an increase in fluorescence. Dodecanoyl-QHSQITKV and Myristoyl-QHSQITKV result in lower critical aggregate concentrations (CAC) that is ˜5-fold lower from their equivalent lipidized peptides with a threonine. This indicates a clearer amphiphilic nature for Dodecanoyl-QHSQITKV and Myristoyl-QHSQITKV and a higher propensity for self-assembly which is explained based on higher water solubility of Dodecanoyl-QHSQITKV vs Dodecanoyl-QHTQITKV and Myristoyl-QHSQITKV vs Myristoyl-QHTQITKV. CAC is ˜20-fold smaller for myristoylated peptides compared to dodecanoyl peptides. Analysis by transmission electron microscopy of 400 μM aqueous dispersions in PBS (7.4, no calcium or magnesium) demonstrated oligomers and aggregates of approximately 20 nm in size that are electron dense. This conserved structure can explain the increased stability and likely the success in solubilizing/stabilizing the peptide aggregates in hydroxyl-b-propyl cyclodextrin.

### Example 9

### Summary of Results

Myristoyl-QHTQITKV possess poor physicochemical properties for successful development. Myristoyl-QHSQITKV shows poor BBB permeability. From the tested peptides, myristoyl-QHSQITKV, dodecanoyl-QHSQITKV, and dodecanoyl-QHTQITKV can be successfully developed for intravenous, subcutaneous and likely nasal delivery. However, only Dodecanoyl-QHSQITKV and Dodecanoyl-QHTQITKV show both good BBB permeability and cell viability, together with good human plasma stability.

Based on the studies presented herein, dodecanoyl-QHSQITKV and dodecanoyl-QHTQITKV are peptide candidates for drug delivery.

Table 6 below presents a summary of the results obtained in the examples as demonstrated herein above.

| | Dodecanoyl- | Dodecanoyl- | Myristoyl- | Myristoyl- |
|---|---|---|---|---|
| | QHTQITKV | QHSQITKV | QHTQITKV | QHSQITKV |
| Plasma stability (mouse) | ++ | +++++ | ++ | +++ |
| Plasma stability (human) | +++++ | +++++ | +++++ | + |
| Brain stability | ++++ | +++ | ++++ | +++ |
| Liver stability | ++ | ++ | ++ | +++++ |
| Simulated intestinal fluid | ++ | + | ++ | +++++ |
| stability | | | | |
| BBB permeability | ++++ | ++++ | ++ | + |
| Cell viability | +++++ | +++++ | +++++ | + |
| Water solubility | ++ | +++++ | ++ | +++++ |

1. A peptide conjugate N-Dodecanoyl-QHTQITKV (conjugate of peptide SEQ ID NO:1), derivative or peptidomimetic thereof.

2. A peptide conjugate N-Dodecanoyl-QHSQITKV (conjugate of peptide SEQ ID NO:2), derivative or peptidomimetic thereof.

3. A method of preventing or treating a β-amyloidogenic disease comprising administering a pharmaceutically effective amount of a peptide conjugate of claim 1, to a subject in need thereof.

4. The method of claim 3, wherein said β-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM).

5. A method of preventing, mitigating, or alleviating synaptic or cognitive deficits associated with a β-amyloidogenic disease comprising administering a pharmaceutically effective amount of a peptide conjugate of claim 1, to a subject in need thereof.

6. The method of claim 5, wherein said β-amyloidogenic disease is Alzheimer's disease, Parkinson's disease (PD), mild cognitive impairment (MCI), multiple sclerosis; HIV-related dementia, ALS (amyotropic lateral sclerosis), or inclusion-body myositis (IBM).

7. A method of preventing or treating Alzheimer's disease comprising administering a pharmaceutically effective amount of a peptide conjugate of claim 1, to a subject in need thereof.

8. A method of treating symptoms of Alzheimer's disease comprising administering a pharmaceutically effective amount of a peptide conjugate of claim 1, to a subject in need thereof.

9. The method of claim 8, wherein said symptoms are mild cognitive impairment or age-associated memory loss.

10. A method of improving age-related memory impairment and enhancing cognitive function in healthy individuals comprising administering a pharmaceutically effective amount of a peptide conjugate of claim 1, to a subject in need thereof.

11. The method of claim 10, wherein said administering is by injection.

12. A composition comprising the peptide conjugate of claim 1 and a pharmaceutically acceptable carrier.
